(19) United States
(12) Patent Application Publication
(10) Pub. No.: US 2014/0335135 A1
(43) Pub. Date: Nov. 13, 2014

(54) PHARMACEUTICAL COMPOSITION

(71) Applicant: FERRING BV, Hoofddorp (NL)

(72) Inventors: Michael Moller, St-Cergue (CH); Robert Gurny, Geneva (CH); Asmus Lutz, Freiburg im Breisgau (DE); Gregoire Schwach, Copenhagen (DK)

(73) Assignee: FERRING BV, Hoofddorp (NL)

(21) Appl. No.: 14/362,530

(22) PCT Filed: Dec. 5, 2012
(86) PCT No.: PCT/EP2012/074448

§ 371 Date: Jun. 3, 2014

(30) Foreign Application Priority Data
Dec. 5, 2011 (EP) 11191942.9

(51) Int. Cl. A61K 47/34; A61K 9/00; A61K 38/09

(52) U.S. Cl. CPC A61K 47/34; A61K 38/09; A61K 9/0019
USPC 424/400; 514/10.3

(57) ABSTRACT

Sustained release formulations of triptorelin are provided.

The present invention relates to pharmaceutical compositions, for example sustained release compositions.

Triptorelin is a gonadotropin-releasing hormone agonist (GnRH agonist). Triptorelin may be used in the treatment of hormone-responsive cancers such as breast cancer or prostate cancer; in the management of endometriosis, female infertility and uterine fibroids; and in treatment of precocious puberty.

Triptorelin is a decapeptide having the formula pGlu-His-Trp-Ser-Tyr-D-Trp-Leu-Arg-Pro-Gly-NH₂. The systematic (IUPAC) name is L-Pyroglutamyl-L-histidyl-Ltryptophyl-L-seryl-L-tyrosyl-D-tryptophyl-L-eucy-L-arginy-L-prolyl-glycinamide, or [D-Trp6]GnRH. Triptorelin is marketed as a pharmaceutically acceptable salt (e.g. acetate or pamoate), for example under the names Decapeptyl® and Pamorelin® LA.

Treatment using triptorelin is generally long term, and it is advantageous to provide sustained release or depot formulations, which reduce the requirement to visit medical practitioners (for administration) and therefore improve patient compliance. Current triptorelin preparations include depot formulations which provide sustained release over three months. However, products such as Decapeptyl® are formulated as powders for suspension for injection, which require re-suspension and two chamber syringes for administration. Further, the known sustained release preparations utilise PLGA. Thus, most of these formulations require storage under cooled conditions to reduce the possibility of polymer degradation in storage and any resulting change of release characteristics. The known formulations include formulations based on microencapsulation technology; the production of such microcapsules is complicated and further improvements are desirable.

There is therefore a need for alternative and/or improved formulations of GnRH analogs, for example GnRH agonists, for example triptotrelin, which provide effective stability and/or sustained release, and/or satisfactory/Improved administration (e.g. injection) characteristics. For example, there is a need for formulations of GnRH analogs, for example GnRH agonists, for example triptorelin, which are directly injectable (i.e. which do not require resuspension etc. prior to administration). There is also a need for formulations of GnRH analogs, for example GnRH agonists, for example triptorelin, which are room temperature stable (for example at 25° C. and 60% relative humidity) for up to two years.

According to the present invention there is provided a pharmaceutical composition comprising a liquid viscous alkyl substituted polylactide; and a GnRH analog or a pharmaceutically acceptable salt or derivative thereof. Preferably the GnRH analog is a GnRH agonist. Preferably the GnRH analog is triptorelin. The liquid, viscous, alkyl substituted polylactide may be a C₅-C₁₁ alkyl substituted polylactide, preferably a C₆ alkyl substituted polylactide. The polylactide may be a hexyl substituted polylactide. Preferably, the pharmaceutical composition comprises micronised triptorelin or pharmaceutically acceptable salt or derivative thereof. The composition may be for use in the treatment of hormone-responsive cancers such as breast cancer or prostate cancer; In the management of endometriosis, female infertility and uterine fibroids; and in treatment of precocious puberty.

Thus, the present invention may provide a pharmaceutical composition comprising a C₅-C₁₁ alkyl substituted polylactide (e.g a C₆ alkyl substituted polylactide); and triptorelin or a pharmaceutically acceptable salt or derivative thereof.

Herein, the liquid, viscous, alkyl substituted polylactide may be a C₅-C₁₁ alkyl substituted polylactide, preferably a C₆ alkyl substituted polylactide. The liquid, viscous, alkyl substituted polylactide may be a C₅ alkyl substituted polylactide, a C₆ alkyl substituted polylactide, a C₇ alkyl substituted polylactide, C₈ alkyl substituted polylactide, C₉ alkyl substituted polylactide, a C₁₀ alkyl substituted polylactide, or a C₁₁ alkyl substituted polylactide.

The use of polylactides in pharmaceutical formulations known, for example, from WO2007/012979.

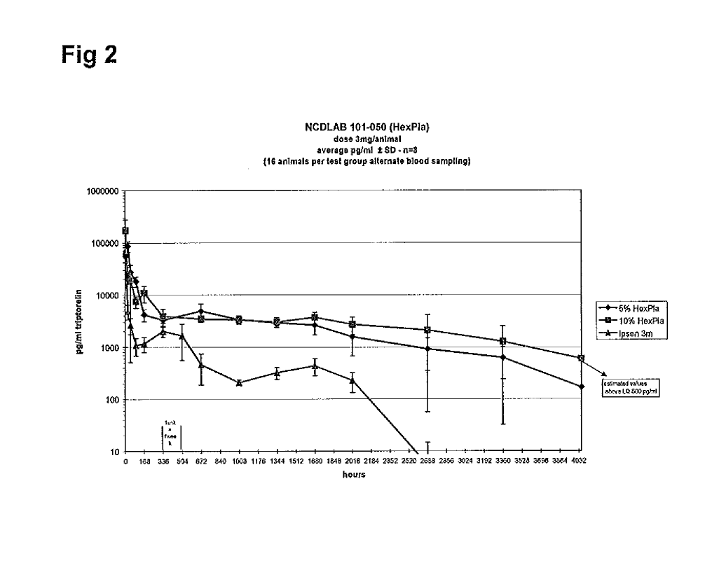

However, the present applicants have surprisingly found that the use of a liquid, viscous, alkyl substituted polylactide, for example a hexyl substituted polylactide, for example a mono- or di-hexyl substituted polylactide, together with (e.g. micronized) triptorelin (or salt or derivative thereof), may provide a homogeneous or substantially homogeneous pharmaceutical composition which has markedly improved sustained release (up to, for example, six months) and improved injection characteristics (FIG. 2, Example 2). The present applicants have also surprisingly found that the use of a liquid, viscous, alkyl substituted polylactide, for example a hexyl substituted polylactide, for example a mono- or di-hexyl substituted polylactide, together with (e.g. micronized) triptorelin (or salt or derivative thereof), may provide a homogeneous or substantially homogeneous pharmaceutical composition which is directly injectable (i.e. which does not require resuspension etc. prior to administration), and/or which is room temperature stable (for example at 25° C. and 60% relative humidity) for up to two years.

Micronised triptorelin is triptorelin which has been subject to micronization to reduce the average diameter of its solid particles. Herein, the term “micronised” means triptotrelin (or other solid) which has average particle diameter on a micrometer or nanometer, preferably micrometer, scale. Preferably, the pharmaceutical composition comprises particles including triptorelin or a pharmaceutically acceptable salt or derivative thereof, the particles having an average particle diameter of 0.01 μm to 900 μm. Preferably the particles have average particle diameter (for example, for the coextensive circle) of 0.5 to 11 μm, for example 2.0 to 9.5 μm, more preferably 3 μm to 10 μm, more preferably 3.5 μm to 5 μm. The applicants have found that particles (which include triptorelin) having average particle diameter (for example, for the coextensive circle) of about 0.5 μm to 5 μm, for example 3.5 μm to 5 μm, e.g. formed by a combined cryo-milling-mixing process as set out below, may provide a formulation having a reduced burst release. This means these particles provide a reduced release of active substance immediately after administration, thereby limiting the initial release of active substance more closely to that required for therapeutic activity; this initial retention of the active substance also means that more is available for sustained release.

The liquid, viscous, alkyl substituted polylactide, for example, a C₅-C₁₁ alkyl substituted polylactide, for example a hexyl substituted polylactide, for example a mono- or di-hexyl substituted polylactide, may be mixed (e.g admixed, dispersed with, used as a pharmaceutically acceptable carrier for) the triptorelin or a pharmaceutically acceptable salt or derivative thereof. The composition may comprise additional components in, or as, the pharmaceutically acceptable carrier.

The weight average molecular weight of the liquid, viscous, alkyl substituted polylactide, for example, C₅-C₁₁ alkyl substituted polylactide (e.g. hexyl substituted polylactide) in the composition may be from 1,000 to 10,000 g/mol, for example 1,200 to 7,500 g/mol.

In some examples of the invention, the weight average molecular weight of the liquid, viscous, alkyl substituted polylactide (e.g. hexyl substituted polylactide) in the composition may be 2,000 to 6,000 g/mol, preferably 2,100 to 5,100 g/mol, preferably 2,200 to 3,000 g/mol. The applicants have found that compositions including a liquid, viscous, alkyl substituted polylactide (e.g. hexyl substituted polylactide) having a weight average molecular weight of 2,000 to 6,000 g/mol, e.g. 2, 500 or 5,000 g/mol may provide a longer sustained release. Presently compositions including a liquid, viscous, alkyl substituted polylactide (e.g. hexyl substituted polylactide) having a weight average molecular weight of 2,200 to 3,000 g/mol, e.g. 2500 g/mol, are preferred because of combination of release profile and injectibility (viscosity—see below).

In some examples of the invention, the weight average molecular weight of the liquid, viscous, alkyl substituted polylactide (e.g. hexyl substituted polylactide) in the composition may be from 1,000 to 7,500 g/mol. The applicants have found that compositions having lower weight average molecular weight have low viscosity which may improve injection characteristics e.g. administration through a narrow (for example a 21 G) needle. However, the applicants have found that compositions having lower weight average molecular weight may give a higher initial “burst” (i.e. higher initial systemic concentration of active agent post administration) which may be associated with reduced sustained release capability.

In some examples of the invention, the weight average molecular weight of the liquid, viscous, alkyl substituted polylactide (e.g. hexyl substituted polylactide) in the composition may be 2,750 to 10,000 g/mol. The applicants have found that compositions including a higher weight average molecular weight liquid, viscous, alkyl substituted polylactide (e.g. hexyl substituted polylactide), for example a weight average molecular weight of 2750 g/mol or higher, may be viscous and therefore administration through a narrow (for example a 21 G) needle may be difficult. Compositions of the Invention may therefore further comprise a plasticiser (e.g. to reduce viscosity (e.g. lower the glass transition temperature) and/or improve injection characteristics).

The pharmaceutical composition may comprise a plasticiser. Preferably the plasticiser is an FDA approved plasticiser, such as are well known in the art. The plasticiser may be for example benzyl benzoate, cellulose acetate etc.

The pharmaceutical composition may also comprise a solvent of the polymer (e.g. to reduce the viscosity and/or improve injection characteristics). The solvent may further improve the drug release characteristics (e.g. by solvent displacement after injection), especially if the solvent is also a solvent of the GnRH analog (e.g. co-solvent). A co-solvent of the polymer and the GnRH analog may be used to improve the drug dispersion in the formulation and improve the injection characteristics and/or the release characteristics. The co-solvent may help to solubilise the drug in the formulation and may yield to a solution for injection rather than a suspension thus improving the injection and/or the release characteristics.

The pharmaceutical composition may comprise a solvent of the polymer. The solvent may also be a solvent of the GnRH analog (co-solvent). Preferably the solvent is an FDA approved solvent for injectables, such as are well known in the art. The solvent may be for example dimethylsulfoxide (DMSO), N-methyl pyrolidone (NMP), ethyl acetate etc.

Preferably the weight average molecular weight of the liquid, viscous, alkyl substituted polylactide (e.g. hexyl substituted polylactide) in the composition is 1,800 g/mol or greater. The applicants have found that compositions of the invention including the polylactide having weight average molecular weight of 1,800 g/mol or greater may allow long term storage at room temperature.

Preferably the liquid, viscous, alkyl substituted polylactide (e.g. hexyl substituted polylactide) is viscous. Herein, the term “viscous” is used to define a polylactide that has a glass transition temperature (Tg) value of less than 44° C. Preferably, the liquid, viscous, alkyl substituted polylactide (e.g. hexyl substituted polylactide) has a glass transition temperature (Tg) value of less than 36° C., more preferably less than 0° C., more preferably less than −10° C. In an example, the liquid, viscous, alkyl substituted polylactide (e.g. hexyl substituted polylactide) has a glass transition temperature (Tg) value of from −10° C. to −20° C., e.g. −17° C. Preferably, the pharmaceutical composition has a viscosity of 5 to 70 Pa×s, for example 15 to 65 Pa×s, for example 15 to 35 Pa×s, at a temperature of 20° C. and/or a shear rate of 100 1/s or less.

In an example, the composition has a viscosity of 21 Pa×s or greater, for example 21 to 50 Pa×s. The applicants have found that compositions having a viscosity of 21 Pa×s or greater have a multiple phase release profile which may provide superior sustained release of active ingredient (e.g triptorelin).

The pharmaceutical composition may comprise 2 to 15% by weight triptorelin or a pharmaceutically acceptable salt or derivative thereof, preferably 2.5 to 15% by weight triptorelin or a pharmaceutically acceptable salt or derivative thereof preferably 4 to 12% by weight triptorelin or a pharmaceutically acceptable salt or derivative thereof, more preferably 5 to 10% by weight triptorelin or a pharmaceutically acceptable salt or derivative thereof. Presently compositions including 4 to 12% by weight triptorelin, e.g. 5% or 10% by weight triptorelin, are preferred because they have a superior sustained release profile.

The pharmaceutical composition(s) according to the invention may be formed (prepared) by a method comprising micronising the triptorelin or pharmaceutically acceptable salt thereof and mixing with the liquid, viscous, alkyl alkyl substituted polylactide (e.g. C₅-C₁₁ alkyl substituted polylactide, e.g. hexyl substituted polylactide). The method may comprise micronising the triptorelin or pharmaceutically acceptable salt thereof and mixing the triptorelin or pharmaceutically acceptable salt or derivative thereof with the polylactide. Preferably, the micronising is by cryo-milling the triptorelin or a pharmaceutically acceptable salt or derivative thereof. Herein, the term “cryo-milling” means milling at a low temperature, for example a temperature of −196° C. in liquid nitrogen. The composition may be formed by micronizing (e.g. cryo-milling) the triptorelin or pharmaceutically acceptable salt or derivative thereof prior to mixing with the polylactide. The composition may be formed by a single step of micronising the triptorelin or pharmaceutically acceptable salt or derivative together with the polylactide. Preferably the single step comprises cryo-milling the triptorelin or a pharmaceutically acceptable salt or derivative thereof together with the polylactide. The composition may be formed by micronizing (e.g. cryo-milling) the triptorelin or pharmaceutically acceptable salt or derivative thereof; micronizing (e.g. cryo-milling) the polylactide; and mixing the micronized triptorelin and the micronized polylactide.

The pharmaceutical composition(s) according to the invention may therefore be formed (prepared) by micronising the triptorelin or pharmaceutically acceptable salt or derivative thereof with the polylactide in a single step (e.g. In a single step of cryomilling the triptorelin or pharmaceutically acceptable salt or derivative thereof (together) with the polylactide). Preferably the triptorelin (or salt/derivative thereof) is micronized/mixed with the polylactide at a temperature below the glass transition temperature of the polylactide, more preferably at temperature of around −196° C., so as to avoid melting and resulting increased viscosity of the polylactide. The applicants have surprisingly found that such a “direct cryo-milled formulation” or “cryo-milled-mixed” formulation is more homogeneous than, and/or has a reduced average particle diameter (of about 3.5 μm to 5 μm) compared with, formulations formed by separate micronization and mixing steps; the cryo-milled-mixed formulations may show a less pronounced burst during the first three weeks, which is more advantageous for controlled release (see Example 1). Pharmaceutical compositions prepared in this way may be distinguished over those of the prior art (or those which are prepared in other ways) because they are more homogeneous and/or have particle size of about 3.5 μm to 5 μm.

According to the present invention in a further aspect there is provided a process for preparation of a pharmaceutical composition comprising a liquid, viscous, alkyl substituted polylactide (e.g. C₅-C₁₁ alkyl substituted polylactide, e.g. hexyl substituted polylactide); and triptorelin or a pharmaceutically acceptable salt or derivative thereof; the process comprising micronising the triptorelin or pharmaceutically acceptable salt or derivative thereof and mixing the triptorelin or pharmaceutically acceptable salt or derivative thereof with the polylactide. Preferably, the micronising is by cryo-milling the triptorelin or a pharmaceutically acceptable salt or derivative thereof. In one example, the triptorelin or pharmaceutically acceptable salt or derivative thereof is micronized (e.g. cryo-milled) prior to mixing with the polylactide. Preferably, however, the process comprises a single step of micronising the triptorelin or pharmaceutically acceptable salt or derivative thereof during mixing with the polylactide. Preferably the single step comprises cryo-milling the triptorelin or a pharmaceutically acceptable salt or derivative thereof together with the polylactide. The process may comprise micronizing (e.g. cryo-milling) the triptorelin or pharmaceutically acceptable salt or derivative thereof, micronizing (e.g. cryo-milling) the polylactide; and mixing the micronized triptorelin and the micronized polylactide. Preferably the triptorelin (or salt/derivative thereof) is micronized/mixed with the polylactide at a temperature below the glass transition temperature of the polylactide, more preferably at temperature of around −196° C., so as to avoid melting and resulting increased viscosity of the polylactide.

According to the present invention in a further aspect there is provided a pharmaceutical composition, for example a room temperature stable pharmaceutical composition, comprising a liquid, viscous, alkyl substituted polylactide (e.g. C₅-C₁₁ alkyl substituted polylactide, e.g. hexyl substituted polylactide); and a GnRH analog or a pharmaceutically acceptable salt or derivative thereof. The GnRH analog may be a GnRH agonist or antagonist. The GnRH analog may be triptorelin. The polylactide may be a hexyl substituted polylactide. Preferably, the pharmaceutical composition comprises micronised GnRH analog or pharmaceutically acceptable salt or derivative thereof.

Preferably the composition is a room temperature stable pharmaceutical composition which remains stable at 25° C. and 60% relative humidity for three months (84 days) or longer.

Herein the term “room temperature stable” means a composition which is stable (little or no change in composition) at 25° C. and 60% relative humidity for up to two years, The applicants have surprisingly found that compositions of the invention may provide room temperature stable formulations of GnRH analogs (see Example 3).

The (for example room temperature stable) pharmaceutical composition(s) according to the invention may be formed (prepared) by a method comprising micronising the GnRH analog (e.g. GnRH agonist, e.g. triptorelin) or pharmaceutically acceptable salt or derivative thereof and mixing with the liquid, viscous, alkyl substituted polylactide (e.g. C₅-C₁₁ alkyl substituted polylactide, e.g. hexyl substituted polylactide). The method may comprise micronising the GnRH analog (e.g. GnRH agonist, e.g. triptorelin) or pharmaceutically acceptable salt or derivative thereof and mixing the micronized GnRH analog (e.g. GnRH agonist, e.g. triptorelin) or pharmaceutically acceptable salt or derivative thereof with the polylactide. Preferably, the micronising is by cryo-milling the GnRH analog (e.g. GnRH agonist, e.g. triptorelin) or a pharmaceutically acceptable salt or derivative thereof. Thus, the composition may be formed by micronizing (e.g. cryo-milling) the triptorelin or pharmaceutically acceptable salt or derivative thereof prior to mixing with the polylactide. Preferably, however, the composition is formed by a single step of micronising the GnRH analog (e.g. GnRH agonist, e.g. triptorelin) or pharmaceutically acceptable salt or derivative thereof while mixing the GnRH analog (e.g. GnRH agonist, e.g. triptorelin) or pharmaceutically acceptable salt or derivative thereof with the polylactide. Preferably the single step comprises cryo-milling the GnRH analog (e.g. GnRH agonist, e.g. triptorelin) or a pharmaceutically acceptable salt or derivative thereof together with the polylactide. The method may comprise micronising the GnRH analog (e.g. GnRH agonist, e.g. triptorelin) or pharmaceutically acceptable salt or derivative thereof and, separately, micronising the polylactide; and a subsequent step of mixing the micronized GnRH analog (e.g. GnRH agonist, e.g. triptorelin) or pharmaceutically acceptable salt or derivative thereof with the micronized polylactide.

According to the present invention in a further aspect there is provided the use in the manufacture of a medicament for the treatment of hormone-responsive cancers such as breast cancer or prostate cancer; for the management of endometriosis, female infertility and uterine fibroids; and for the treatment of precocious puberty, comprising a liquid, viscous, alkyl substituted polylactide (e.g. C₅-C₁₁ alkyl substituted polylactide, e.g. hexyl substituted polylactide); and a GnRH analog or a pharmaceutically acceptable salt or derivative thereof.

Preferably the GnRH analog is a GnRH agonist. Preferably the GnRH analog is triptorelin. The polylactide may be a hexyl substituted polylactide. Preferably, the pharmaceutical composition comprises micronised triptorelin or pharmaceutically acceptable salt or derivative thereof.

According to the present invention in a further aspect there is provided a method of treatment of hormone-responsive cancers such as breast cancer or prostate cancer; a method of treatment or management of endometriosis, a method of treatment of female infertility, a method of treatment of uterine fibroids; and/or a method of treatment of precocious puberty; comprising a step of administering to a patient in need thereof a pharmaceutically effective amount of a composition comprising a liquid, viscous, alkyl substituted polylactide (e.g. C₅-C₁₁ alkyl substituted polylactide, e.g. hexyl substituted polylactide); and a GnRH analog or a pharmaceutically acceptable salt or derivative thereof. Preferably the GnRH analog is a GnRH agonist. Preferably the GnRH analog is triptorelin. The polylactide may be a hexyl substituted polylactide. Preferably, the composition comprises micronised triptorelin or pharmaceutically acceptable salt or derivative thereof. The method may include a further step of warming the composition to above 25° C. prior to administration to the patient.

The pharmaceutical composition(s) of the Invention may be formulated into compositions for any well known route of drug administration, e.g. oral, rectal, parenteral, transdermal (e.g. patch technology), intravenous, intramuscular, subcutaneous, intrasusternal, intravaginal, intraperitoneal, local (powders, ointments or drops), an aerosol, or as a buccal or nasal spray. Preferably, the pharmaceutical composition(s) of the invention is formulated for injection. A typical composition may further comprise a pharmaceutically acceptable carrier, such as a solution, non toxic excipients, including salts, sugars, amino acids, surfactants, preservatives, stabilisers, isotonicity agents, buffers and the like, as described in Remington's Pharmaceutical Sciences fifteenth edition (Matt Publishing Company, 1975), at pages 1405 to 1412 and 1461-87, and the national formulary XIV fourteenth edition (American Pharmaceutical Association, 1975), among others. Examples of suitable aqueous and non-aqueous pharmaceutical carriers, diluents, solvents or vehicles include water, ethanol, polyols (such as glycerol, propylene glycol, polyethylene glycol, and the like), carboxymethylcellulose and suitable mixtures thereof, vegetable oils (such as castor oil), and Injectable organic esters such as ethyl oleate. Non-aqueous pharmaceutical carriers and solutions may be preferred. The products (and doses and compositions) of the present invention also can contain additives such as but not limited to preservatives, wetting agents, emulsifying agents, and dispersing agents. Antibacterial and antifungal agents can be included to prevent growth of microbes and includes, for example, paraben, chlorobutanol, phenol, sorbic acid, and the like. Furthermore, it may be desirable to include isotonic agents such as sugars, sodium chloride, and the like.

Injectable formulations and compositions can be sterilized, for example, by filtration through a bacterial-retaining filter, or by incorporating sterilizing agents in the form of sterile solid compositions which can be dissolved or dispersed in sterile water or other sterile injectable medium just prior to use. Injectable formulations may be prepared using aseptic preparation methods. Injectable formulations can be supplied in any suitable container, e.g. vial, pre-filled syringe, injection cartridges, and the like.

The pH and exact concentration of the various components of the product are adjusted in accordance with routine practice in this field. See GOODMAN and GILMAN's THE PHARMACOLOGICAL BASIS FOR THERAPEUTICS, 7^{th} ed. In a preferred embodiment, the products of the invention are supplied as compositions for parenteral administration. General methods for the preparation of the parenteral formulations are known in the art and are described in REMINGTON; THE SCIENCE AND PRACTICE OF PHARMACY, supra, at pages 780-820. The parenteral products can be supplied in liquid formulation or as a solid which will be mixed with a sterile injectable medium just prior to administration. In an especially preferred embodiment, the parenteral products are supplied in unit dosage form for ease of administration and uniformity of dosage.

### DETAILED DESCRIPTION OF THE INVENTION

The present invention will be now described in further detail, with reference to the following drawings in which:

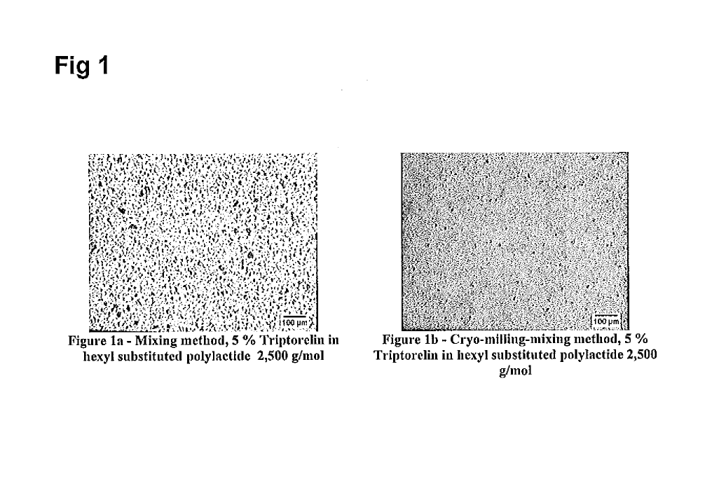

FIG. 1 shows microscopy pictures of triptorelin formulations comprising 5% by weight triptorelin in a hexyl substituted polylactide having weight average molecular weight 2,500 g/mol formed by the “separate milling and mixing” method (FIG. 1a), and formed by the “cryomilling and mixing” method (FIG. 1b);

FIG. 2 shows in vivo release results of formulations containing 5% and 10%, respectively, triptorelin together with a hexyl substituted polylactide having weight average molecular weight of 2500 g/mol; compared to the prior art “Ipsen 3 month” formulation;

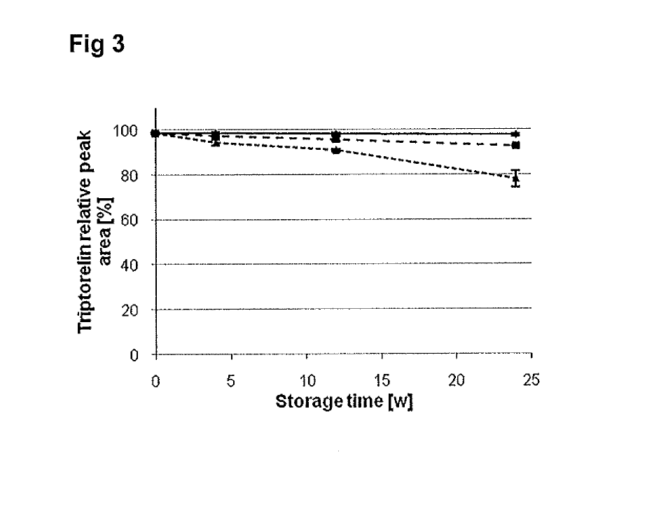

FIG. 3 shows stability of a formulation including 10% triptorelin together with a hexyl substituted polylactide having weight average molecular weight 2500 g/mol over six months (about 24 weeks) at 5° C. (solid line), 25° C. (dashed line), and 40° C. (dotted line);

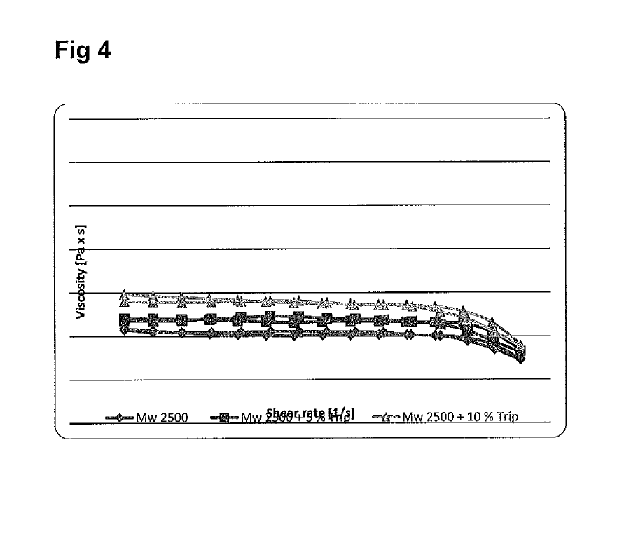

FIG. 4 shows the viscosity of formulations including 0%, 5% and 10% triptorelin together with a hexyl substituted polylactide having weight average molecular weight of 2500 g/mol; and

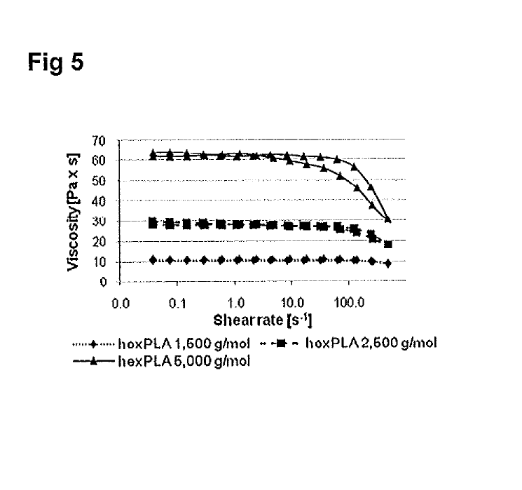

FIG. 5 shows the rheological behavior of hexyl substituted polylactide formulations with different molecular weights containing 10% triptorelin at a constant temperature of 20° C. (“hexPLA” is hexyl substituted polylactide).

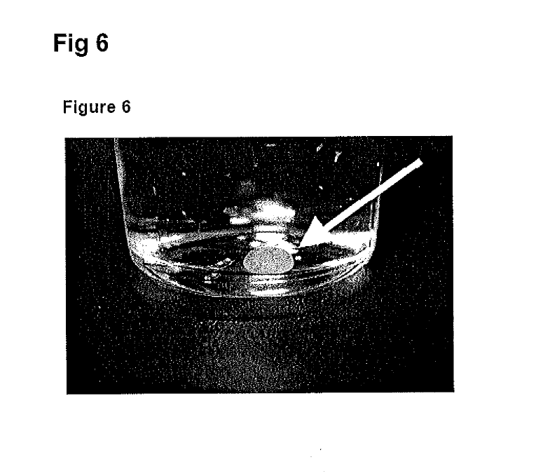

FIG. 6 shows a photo of an inherently stable, hydrophobic formulation droplet in KRT-buffer;

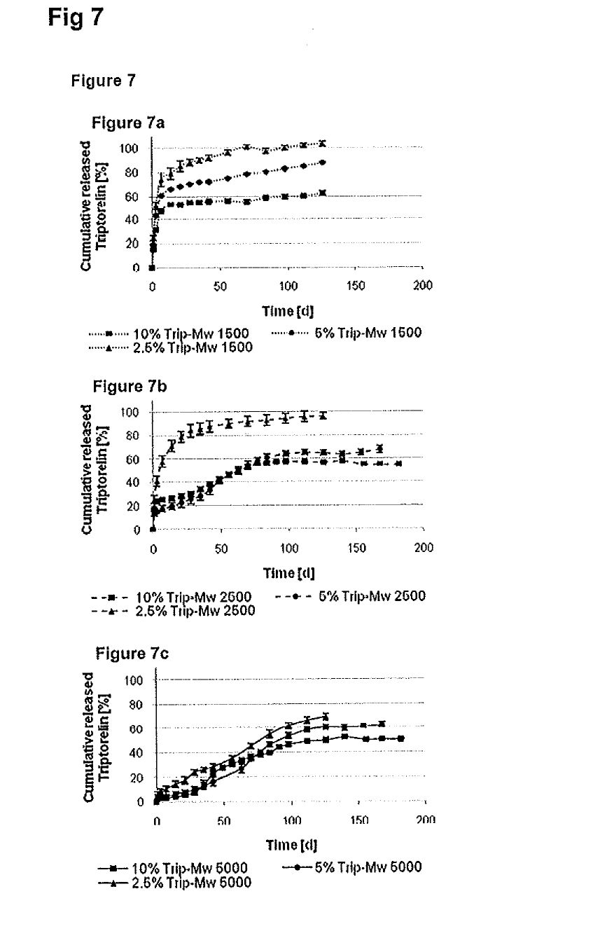

FIG. 7 shows in vitro release profiles over time (d=days) of nine different formulations with 2.5% (▴), 5% (•) and 10% (▪)triptorelin in polymer of 1,500 g/mol (dotted line), 2,500 g/mol (dashed line) and 5,000 g/mol (solid line);

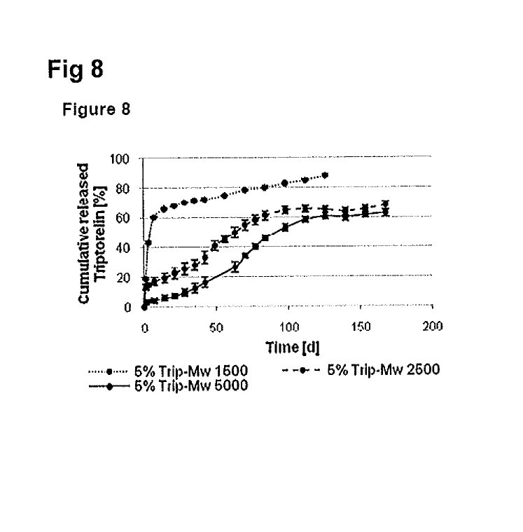

FIG. 8 shows comparison over time (d=days) of three triptorelin-hexyl substituted polylactide formulations with the same loading but different molecular weights in vitro; and

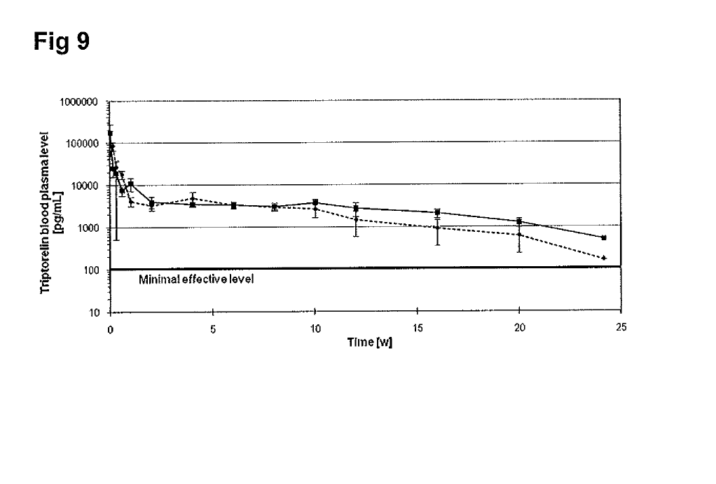

FIG. 9 shows in vivo plasma data of triptorelin released from triptorelin in hexyl substituted polylactide formulations with polymer of molecular weight 2,500 g/mol over six months (24 weeks) (solid line=10% triptorelin, dashed line=5% triptorelin) in SPF Wistar Hannover rats, eight animals per time point.

The present invention provides pharmaceutical compositions comprising an alkyl substituted polylactide; and triptorelin.

The use of polylactides in pharmaceutical formulations is known from WO 2007/012979. This document discloses alkyl substituted polylactides having the structure below

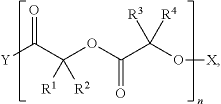

wherein R¹, R², R³, and R⁴, are each independently chosen from the group consisting of alkyl (e.g. unsubstituted alkyl), H, alkenyl and alkyl aryl; X is hydrogen, —C(O)—CH—CH2 or other functional or crosslinking group; and Y is selected from —OH, alkoxy, benzyloxy and —O—(CH₂—CH₂—O)ₚ—CH₃ where p is 1 to 700. The term n is an integer from 1 to 500 or more, more preferably 1 to 100, more preferably 1 to 50, more preferably 1 to 25. The term n may be for example, from 1 to 12, e.g. 1, 2, 3, 4, 5, 6, 7, 8, 9 or 10. In some embodiments, R¹, R³, are hydrogen and R² and R⁴ are C₁-C₁₁ alkyl.

The present applicants have found that the combination of triptorelin or a pharmaceutically acceptable salt or derivative thereof with an alkyl substituted polylactide of the formula above may provide a pharmaceutical composition which displays remarkable sustained release in vivo, for example a sustained release of double or more that of the currently available formulation. The applicants have found that compositions in which R¹ and R³ are hydrogen and R² and R⁴ are C₁-C₁₁ alkyl (for example where R² is methyl and R⁴ is hexyl, or R² is hexyl and R⁴ is methyl, or both R² and R⁴ are hexyl) may be particularly effective. Hexyl-substituted poly(lactic) acid is viscous liquid at toom temperature and slowly degradable in the presence of water. The polyester backbone means that it is subject to hydrolysis in an aqueous environment.

### EXAMPLE 1

### Preparation of Samples

Triptorelin acetate was obtained from Ferring GmbH (Kiel, Germany), in lyophilised form. The hexyl substituted polylactides used were of the general formula above wherein R¹ and R³ are hydrogen, R² is methyl and R⁴ is hexyl. In other words, the polylactide was a hexyl substituted polylactide with the same backbone as poly(lactic) acid but with hexyl side groups replacing all the methyl groups. The hexyl chains are believed to act as internal plasticisers, reducing the glass transition temperature; the hexyl substituted polylactide is a viscous liquid at room temperature.

The hexyl substituted polylactides used may be synthesised by methods known in the art. For example, the hexyl substituted polylactides may be synthesised by first forming 3-methyl-6-hexyl-1,4-dioxane-2,5-dione by the method set out in Example 1 of WO 2007/012979, and ring opening with Sn(Oct)₂, as also set out in Example 1 of WO 2007/012979. The hexyl substituted polylactides may also be synthesised from heptaldehyde, by the melt condensation method published in Asmus, L. R., R. Gurny, and M. Moller, Solutions as solutions—Synthesis and use of a liquid polyester excipient to dissolve lipophilic drugs and formulate sustained-release parenterals (European Journal of Pharmaceutics and Biopharmaceutics, 79 (2011) 584-591), see also Trimaille, T., R. Gurny, and M. Moller, Poly(hexyl-substituted lactides): Novel injectable hydrophobic drug delivery systems (Journal of Biomedical Materials Research Part A, 2007. 80A(1): p. 55-65). Three hexyl substituted polylactide polymers, having weight average molecular weights of 1,500 g/mol, 2,500 g/mol, and 5,000 g/mol, were synthesised using these methods. It will be appreciated that other methods of preparation of the polymers are available. The polymers were sterilised by a standard dry heat sterilisation technique.

Two different methods were used to incorporate triptorelin into the hexyl substituted polylactide polymer. The first was a “separate milling and mixing” method in which the triptorelin was micronized using an SPEX 6700 Freezer/mill at −196° C. in liquid nitrogen (milling for 5 min), the resulting micronized powder being mixed with the hexyl substituted polylactide polymer by kneading in plastic bags until a homogeneous formulation was obtained (as verified by optical microscopy).

In the second method, lyophilised triptorelin (10% by weight of polymer or 5% by weight polymer or 2.5% by weight polymer) was incorporated into the polymer by a combined direct “cryo-milling-mixing” technique. The triptorelin was micronized together with the polymer in a cryo-mill (SPEX 6700 Freezer/mill) at −196 C for five minutes in liquid nitrogen. The particle size distribution in the formulations was analysed using an Optishot-2 microscope (Nikon, Tokyo, Japan) and ImageJ software.

It will be appreciated that other preparation methods are possible. For example, it is possible to micronise (cryo-mill) the triptorelin separately from the polymer and then mix the resulting micronized triptorelin and micronised polymer, to give a compound which provides sustained release of triptorelin.

FIG. 1 shows microscopy pictures of triptorelin formulations comprising 5% by weight triptorelin in a hexyl substituted polylactide having weight average molecular weight 2,500 g/mol formed by the “separate milling and mixing” method (FIG. 1a), and formed by the “cryomilling and mixing” method (FIG. 1b).

Triptorelin is not soluble in the hexyl substituted polylactide polymer (which is a viscous liquid) because of its relatively high hydrophilicity. The lyophilized triptorelin was therefore micronized, by cryomilling or spray-drying, to ensure small particle size for incorporation into the polymer. Preferably the triptorelin (or salt/derivative thereof) is micronized/mixed with the polylactide at a temperature below the glass transition temperature of the polylactide, more preferably at temperature of around −196° C., so as to avoid melting and resulting increased viscosity of the polylactide.

In the first method the triptorelin powder had an average diameter of 5.6 μm for the coextensive circle, prior to incorporation into the hexPLA by kneading at room temperature. The kneading provided a homogenous suspension (FIG. 1a) although the average diameter increased to 9.0 μm because some larger particles of more than 30 μm were formed due to agglomeration.

The combined “cryomilling-mixing” method provided homogenous formulations as shown in FIG. 1b, but the diameter of the coextensive circle was significantly reduced from an average 9.0 μm to 4.1 μm in comparison to the separate cryo-milling and incorporation method. The smaller particles were more resistant to precipitation and displayed enhanced storage stability at room temperature.

An in vitro comparison of the release behavior of two equal formulations of 5% triptorelin in hexyl substituted polylactide polymer of 2,500 g/mol formed by the two methods (results not shown) indicated that the combined “cryomilling-mixing” method provided a formulation having a reduced “burst release” during the first 3 days of the study. This means these particles provide a reduced release of active substance immediately after administration, thereby limiting the initial release of active substance more closely to that required for therapeutic activity. This may enhance the sustained release.

In the longer term, formulations produced by both methods constantly released the triptorelin. The combined cryo-milling-mixing product demonstrated slightly higher release levels during day 3 and 35 probably due to the improved homogeneity of the formulation. The direct (single step) cryo-milling mixing provides a more homogeneous product formulation than that formed by a two stage micronising then mixing process (see FIG. 1b), and had the further advantage that a process step is removed.

Further, no aerosol or dust particles were formed during the combined process, which is a significant safety advantage in terms of product handling. The applicants found that homogenous suspensions of triptorelin having small particle size and distribution, suspended in a polymer of molecular weight higher than 1,800 g/mol, prepared by the cryomilling-mixing method, may be readily and simply stored at room temperature.

### EXAMPLE 1A

### Preparation of Samples for Injection

5 g of formulation of 5% triptorelin with hexyl substituted polylactide of weight average molecular weight of 2,500 g/mol was prepared by the the direct (single step) cryo-milling mixing method. In a separate (but similar) process 3 g of formulation of 10% triptorelin with hexyl substituted polylactide of weight average molecular weight of 2,500 g/mol was prepared. Each formulation was filed into a syringe (or syringes) under laminar flow conditions.

Samples showed no microbial growth after 24 hours, indicating that the methods of polymer sterilisation and aseptic formulation under laminar flow are appropriate.

### EXAMPLE 2

### In Vivo Release Study

A release study compared the formulations of Example 1 with the Ipsen decapeptyl 3 month formulation. 48 male SPF Wistar Hannover rats (of the stock HanTac:WH from Taconic-M&B A/S, Silkeborg, Denmark). in the weight range of 250-300 g, rats were randomly allocated into three test groups, Groups A, B or C (16 animals per test group).

Animals in Group A were administered 5% triptorelin in hexyl substituted polylactide (2,500 g/mol) at a dose of 3 mg/animal; animals in Group B were administered 10% triptorelin in hexyl substituted polylactide (2,500 g/mol) at a dose of 3 mg/animal; and animals in Group C were administered Ipsen decapeptyl 3 month formulation at a dose of 3 mg/animal. Administration was by subcutaneous bolus injection.

The triptorelin in hexyl substituted polylactide was kept in a 5 ml luer-(lock) syringes which may be stored at room temperature. Prior to administration to animals the syringe was put on a hot-plate at 30° C. Lids of 2.2 ml eppendorf tubes (lock) were filled to the rim with the viscous test compound, and the eppendorf lids kept on the hot plate. Hamilton syringes (1710 TLL) were used for animal dosing. The syringes were filled directly via the luer cone to the maximum, equal to approx 115 μl. A 21Gx1″ needle was fitted to the syringe and the content adjusted to 60 μl, thereafter 2×30 μl or 1×60 μl can be dosed. Dosing (by volume) can be done at ambient temperature; using these syringes no major resistance occurs. The syringe can be refilled for the next administration without any prior cleaning procedure, there is no drying of the polymer. After use the syringes can be cleaned with acetone.

Blood samples were taken from all animals at various time points after administration. Blood is withdrawn into EDTA by retro-orbital bleeding under light CO₂/O₂ anaesthesia. 0.8 mL of blood was withdrawn from each rat at each time point (enough to produce 0.3 mL of EDTA-plasma).

FIG. 2 shows in vivo release results of formulations containing 5% (Group A) and 10% (Group B), respectively, triptorelin together with a hexyl substituted polylactide having weight average molecular weight 2500 g/mol; compared to the prior art “Ipsen 3 month” formulation (Group C). The plasma levels of the formulations of the invention (Groups A and B) are higher than the for Group C, the competitor product including the same amount of API: the formulations of the invention may provide a long release period of about 6 months (4032 days) compared to 3 months (2016 days) for decapeptyl. Furthermore, the formulations of the invention are easily administrable using Hamilton syringes with no adverse side effects or encapsulation at the injection site.

Accordingly, pharmaceutical compositions of triptotrelin according to the invention may provide effective stability and/or sustained release (especially favourable compared to the prior art formulation), and/or satisfactory/improved administration (e.g. injection) characteristics.

### EXAMPLE 3

### Storage Stability Study (According to ICH Guidelines)

A formulation including 10% triptorelin with hexyl substituted polylactide of weight average molecular weight of 2,500 g/mol was prepared by the method of Example 1. The triptorelin was incorporated into the polymer by the combined direct cryo-milling-mixing technique.

Pre-sterilized glass vials were each filled with 50 mg of 10% Triptorelin in hexPLA 2,500 g/mol. A number of these vials were stored at 5±3° C. (light protected: storage under refrigerator conditions); a second set of samples were stored at 25±2° C. (light protected: storage under ambient conditions for climate zones I and II); and a third set were stored at 40±2° C. (light protected: accelerated ICH storage conditions).

Samples from each set were analyzed directly after preparation and after 4, 12 and 24 weeks of storage. Analysis was using UPLC, by methods known in the art. The Triptorelin was qualitatively extracted from the formulation by vortexing the formulation with Krebs-Ringer-Trs pH 7.4 buffer (“KRT buffer”—see below) and separation of the polymer by centrifugation. The relative peak area of Triptorelin in comparison to the peak area of its assumed degradation products was measured using UPLC. The chromatograms of pure KRT buffer, degraded hexPLA and the monomer 2-hydroxyoctanoic acid were also analyzed as references. Methods of analysis of triptorelin by UPLC are well known in the art and available to the skilled man; see for example, section 5.1 below. It will be appreciated that other UPLC methods of analyzing triptorelin are known in the art.

FIG. 3 summarizes the progression of the triptorelin peak area in the storage stability samples in comparison to the total peak area over time. At 5° C. (solid line) the value remains almost 100% over the entire time period of 3 months, showing minimal degradation. At 5° C. the content of the Triptorelin decreased by only one percent during 6 months. For storage testing conditions for climate zone II at 25° C. (dashed line), the drug content remained over the 90% threshold generally used to define the limit of themaximal shelf life for pharmaceutical products. These results are indicative of long-term room temperature stability at these conditions. Although elevated temperatures of 40° C. (dotted line) showed not to be suitable for the storage of these formulations due to a stability decrease to 78%, the formulations are able to withstand higher temperatures of up to 40° C. with less than 25% peptide degradation during 6 months and without changes in the carrier constitution.

The reduction of the percentage of the Triptorelin peak area becomes more pronounced at higher temperatures and longer storage times. Nevertheless, even at the most extreme condition over 90% intact Triptorelin was detected after 3 months, and the results at lower temperatures are indicative of long-term room temperature stability at 5° C. and 25° C.

Krebs-Ringer-Tris buffer pH 7.4 (KRT):

| Substance | [mM] | [g/mol] | for 1 L [g] |
|---|---|---|---|
| NaCl | 120 | 58.44 | 7.013 |
| KCl | 4.8 | 74.55 | 0.358 |
| CaCl₂ | 2.6 | 110.98 | 0.289 |
| MgSO₄ * 7H₂O | 1.2 | 246.48 | 0.296 |
| Tris | 25 | 121.14 | 3.029 |
| NaN₃ | | | 1.000 |

+2N HCl to adjust the pH to 7.4
Tris = Tris(hydroxymethyl) aminomethane

### EXAMPLE 4

### Rheology and Injectability

The rheological behaviour of a formulation, especially a suspension, is important in assessing its suitability for injection. The final formulation has to be sufficiently liquid to allow easy filling of the syringe and ready injection through needles of narrow outer diameter (to cause minimal discomfort). However, the viscosity has to be sufficiently high to prevent precipitation of the active compound during storage or in vivo, and to ensure the formulation remains localized at the injection site, keeping its surface and shape (surface area) constant.

### EXAMPLE 4A

The following refers to Table 1 and FIGS. 4 and 5. Viscosity tests were performed for formulations prepared according to Example 1. The formulations tested included 0%, 5% or 10% triptorelin in polymers having weight average molecular weight of 1500 g/mol, 2,500 g/mol, or 5,000 g/mol.

Rheology was tested using a Bohlin CVO 120 rheometer (Bohlin Instruments, Cranbury, USA) with a parallel-plate set-up (type 20 mm) and a gap of 1 mm, as is well known in the art. The viscosity of the polymer and triptorelin-polymer formulations was investigated at shear rates between 0.01 and 500 1/s, at a constant temperature of 20° C., The delay time was 3 seconds, and the Integration time was 5 seconds. The dependence of temperature and viscosity was then assessed at a shear rate of 0.1 s₋₁ and in a temperature range between 15° C. and 37° C. at measurement intervals of 1° C.

Table 1 summarizes the viscosities in the ideal viscous range of the pure polymers and of the formulations with 5% and 10% triptorelin.

### Table 1

Viscosity in the Newtonian range* of pure hexPLA and of formulations containing 5% and 10% Triptorelin at 20° C. *0.1 to 100 s⁻¹

TABLE 1
Viscosity in the Newtonian range* of pure hexPLA and of
formulations containing 5% and 10% Triptorelin at 20° C.

| | Pure | +5% Triptorelin | | +10% Triptorelin | |
|---|---|---|---|---|---|
| Polymer | polymer | Vis- | Increase | Vis- | Increase |
| molecular | Viscosity | cosity | toward pure | cosity | toward pure |
| weight | [Pa × s] | [Pa × s] | polymer [%] | [Pa × s] | polymer [%] |
| 1,500 g/mol | 7.3 | 8.6 | 17.1 | 10.4 | 41.9 |
| 2,500 g/mol | 20.9 | 23.9 | 14.8 | 29.0 | 38.8 |
| 5,000 g/mol | 43.9 | 49.8 | 13.5 | 63.8 | 45.4 |

*0.1 to 100 s⁻¹

As can be seen in FIG. 4, increasing the amount of incorporated triptorelin increases the viscosity significantly. The difference for the increase from 5% to 10% was greater than that for 0% to 5%. This is usual for suspensions; the flow of the liquid is more hindered (and the viscosity increased) by the increasing number of incorporated particles. Accordingly, the percentage increase in viscosity with increasing loading is similar, and independent of the molecular weight of the hexyl substituted polylactide polymer.

Formulations with hexyl substituted polylactide of 2,500 g/mol showed lower viscosities of 20 to 30 Pa×s for shear rates under 100 1/s at 20° C. (FIG. 4), and some formulations with hexPLA of 1,500 g/mol had viscosity even less than 10 Pa×s, making their application through narrow needles feasible (see Example 4B).

FIG. 5 shows the results for the 10% triptorelin formulations. The formulations with polymer of a molecular weight of 5,000 g/mol showed a viscosity higher than 40 Pa×s for shear rates under 100 1/s at 20° C. Such viscosities may be too high to apply the formulations through 21 G needles on their own; addition of small amounts of plasticizer might be necessary.

The hexyl substituted polylactide polymers and formulations showed a strong influence of temperature on viscosity (data not shown). For example, for formulations with hexyl substituted polylactide of 1500 g/mol the viscosity was reduced to 20% to 30% of the initial value by altering the temperature from 15° C. to 37° C. The high viscosity at low temperatures is believed to inhibit the precipitation even of suspensions with larger particles, when refrigerated, allowing good suspension stability (even of formulations prepared by separate milling and mixing). The reduced viscosity at elevated temperature would improve the injectability and suggests that warming the formulation prior to injection may improve injectability; this is much easier than the resuspension procedure which is often required in preparing the currently marketed microparticle formulations.

### EXAMPLE 4B

### Injectability

The injectability of a formulation containing 10% triptorelin acetate in hexyl substituted polylactide of 2,500 g/mol was tested.

A silanized glass syringe with an Inner diameter of 6.3 mm was filed with with the formulation. The syringe could be connected to a 21 G- or 23 G-needle, each of 25 mm length. These syringes and needles are often used for intramuscular injections. The plunger speed was fixed at 10 mm/min and the necessary force to empty the syringe was measured on a dynamometer HBM Load Type 23H2 (Hottinger Baldwin Messtechnik, Volketswil, Switzerland) fixed in press type RM 50 (Schenck A G, Nänikon, Switzerland). The plunger speed was chosen to simulate an injection time of less than 10 seconds to apply a theoretical sufficient dose of triptorelin for a 3-month treatment.

A maximum of 38 N was needed to eject the formulation through a 21 G-needle at room temperature without pre-heating; a maximum of 130 N was required for the 23 G-needle. Thus, a good direct injectability through the larger needle is possible with the formulations of the invention. This indicates good injectability of this formulation compared to the prior art formulations because this needle is considerably thinner than the needles necessary for the application of solid GnRH-agonist implants on the market and there is no requirement for resuspension or additional warming prior to administration, and/or requirement for plasticizer in the formulation.

### EXAMPLE 4

### Conclusion

The tested formulations show a viscosity depending on the molecular weight of the hexyl substituted polylactide and the drug loading and a rheological behavior with thixotropy and shear-thinning typical for linear polymers (see FIGS. 4 and 5). A sample formulation with an average viscosity was easily injectable with a standard 3 mL-syringe and 21 G-needle, demonstrating easy applicability.

### EXAMPLE 5

### In Vitro Triptorelin Release from Formulations of the Invention

The influence of (i) the molecular weight of the polymer, (ii) the triptorelin loading and (iii) formulation viscosity, on the in vitro release of triptorelin from formulations of the invention was measured. The performance and macroscopic stability of the suspension formulations in hydrophilic medium were observed. The following refers to FIGS. 7 and 8.

The formulations were made according to the single step method of Example 1 above. The nine formulations were:

2.5% triptorelin with hexyl substituted polylactide of weight average molecular weight of 1,500 g/mol
5% triptorelin with hexyl substituted polylactide of weight average molecular weight of 1,500 g/mol
10% triptorelin with hexyl substituted polylactide of weight average molecular weight of 1,500 g/mol
2.5% triptorelin with hexyl substituted polylactide of weight average molecular weight of 2,500 g/mol
5% triptorelin with hexyl substituted polylactide of weight average molecular weight of 2,500 g/mol
10% triptorelin with hexyl substituted polylactide of weight average molecular weight of 2,500 g/mol
2.5% triptorelin with hexyl substituted polylactide of weight average molecular weight of 5,000 g/mol
5% triptorelin with hexyl substituted polylactide of weight average molecular weight of 5,000 g/mol
10% triptorelin with hexyl substituted polylactide of weight average molecular weight of 5,000 g/mol

### 5.1 Triptorelin Quantification by UPLC

The quantification of triptorelin acetate was performed using an Acquity UPLC system (Waters Corporation, Milford, USA) The column was an Acquity UPLC BEH C18, 50×2.1 mm, 1.7 μm (Waters Corporation), with a mobile phase of 80% (V/V) water containing 0.1% trifluoroacetic acid and 20% isopropanol. An Isocratic method was chosen with a flow rate of 0.4 mL/min and a column temperature of 60° C. The analysis was performed by UV-detection at 210 nm. It will be appreciated that other UPLC methods of analyzing triptorelin are known in the art.

Triptorelin standards were prepared with Krebs-Ringer-Tris buffer pH 7.4. The resulting calibration curve was a typical quadratic curve ranging from 1 to 40 ppm with a correlation coefficient above 0.999.

### In Vitro Triptorelin Release

50 mg of each of the nine formulations was placed each into 20 mL of Krebs-Ringer-Tris (KRT) buffer pH 7.4 (120 mM NaCl, 4.8 mM KCl, 2.6 mM CaCl₂, 1.2 mM MgSO₄, 25 mM tris(hydroxymethyl)aminomethane, 0.1% NaN₃, HCl to adjust pH). The samples were incubated at 37° C. with a slight shaking at 60 rpm.

At each sampling point 500 μL of the release medium were removed and the volume replaced by fresh buffer. The triptorelin content in the release medium was directly determined using the quantitative UPLC method above. The release was performed in quintuplicate over a period of more than 4 months, depending on the release kinetics of the formulation. Initial sampling points were fixed on day 1, 3 and 7, followed by sampling a once a week. The sampling was reduced to every two weeks at later stages.

Macroscopic Stability of hexPLA in Hydrophillic Medium

The nine formulations formed single stable droplets following addition of buffer (FIG. 6).

The high lipophilicity of the polymer causes it to minimize the contact area to the hydrophilic buffer, resulting in a spherical droplet. This means that the formulations independently created similar surface areas, allowing direct comparison of different release samples. The spherical form was maintained through the experiment, although the droplets did shrink with time due to degradation of the polymer on the surface.

This tendency to reduce the surface area has interesting implications for in vivo administration because it suggests the formulations stay coherent at the site of injection.

### Sustained-Release Behaviour

The results are summarized in FIG. 7. All formulations, independently of their loading and polymer molecular weight, showed sustained release of the triptorelin from the polymer matrix.

The duration of in vitro release was at least three months for all formulations. All formulations showed a small standard deviation between the quintuplicate samples in the amount of triptorelin released (generally less than ±5%), even after long release periods, supporting the reproducibility. This is believed to be due to the tendency of the polymer to minimize its surface area, ensuring the different samples are comparable with time.

The nine formulations may be grouped into two release types. The three formulations with a molecular weight of 1,500 g/mol (FIG. 7a) and the formulation with 2.5% Triptorelin in polymer of 2,500 g/mol (FIG. 7b) showed biphasic release; a pronounced burst of 50% to 80% during the first two weeks followed by a phase of slow, constant release. The formulations with 5% and 10% triptorelin in hexyl substituted polylactide polymer of 2,500 g/mol (FIG. 7b) and all formulations with hexyl substituted polylactide polymer of 5,000 g/mol (FIG. 7c) showed multiphase release. They demonstrated a lower burst of less than 25% in the first phase during the initial 14 days, followed by a period of slow release until day 40 to 50. In a third phase, the rate of release increased and lasted until day 90 to 110. Finally, the total release reached a plateau with only small amounts of active substance being released over time.

The formulations with polymer of 2,500 g/mol and 5,000 g/mol showed a favorable ratio between burst and continuous, long term release.

### Influence of Viscosity on the Release-Type and -Rate

The different release types are believed to result from viscosity differences. The four formulations with the lowest dynamic viscosities showed biphasic release behavior, whereas formulations with viscosities higher than 21 Pa s had a multiple phase characteristics.

The low burst values for the more viscous formulations suggests that only that portion of the active substance which was immediately accessible by the buffer on and near the surface was released and dissolved during the first days. The formulations with low viscosities allow rapid diffusion of the active substance from the core to the shell of the sphere; more of the active triptorelin is therefore able to contact the buffer solution and is available for release during the first days. The rapid decrease in release after the burst phase is due to the relatively small amount of triptorelin which remains in the polymer matrix.

The influence of the viscosity on the release from the low molecular weight formulations can in detail be seen in FIG. 7a. The three formulations with polymer of 1,500 g/mol differ only in drug loading and therefore in viscosity: the higher the drug loading the higher the viscosity. FIG. 7a shows the release profiles are similar, but the 21% difference in viscosity between the formulations with 5% and 10% Triptorelin resulted in a reduction in release by over 20% after 90 days. Thus, the rate of release for low molecular weight peptide/polymer appears to be controllable by the viscosity of the suspension, whereas the effect is minor for higher molecular weight formulations.

### Influence of Drug Loading

The drug loading influences the release profile of low molecular weight formulations because it appears to affect the viscosity.

The most pronounced effect of drug loading on the release was observed for the formulations with polymer of 2,500 g/mol these showed very different release profiles (FIG. 7b). The formulation with the low drug loading of 2.5% had a biphasic sustained release but with a longer and more fluent transfer from burst to slow, continuous release, than the formulations with 1,500 g/mol polymer. However, formulations with 5% and 10% active substance in polymer of 2,500 g/mol showed multiphase release behavior. Their release profiles were similar and the differences in released triptorelin much smaller than observed for the corresponding triptorelin suspensions in polymer of 1,500 g/mol. Thus, the influence of loading and viscosity on release rate appears to decrease with higher values for both factors. This assumption was confirmed by the results displayed in FIG. 7c, showing multiphase release profiles of the formulations with polymer of 5,000 g/mol. AN three formulations showed similar release characteristics with the 2.5% formulation releasing the incorporated active substance only slightly faster than the formulations with 5% and 10%. Even after 100 days of release the relative difference between the fastest and slowest releasing formulation was still less than 20%. Thus, for higher molecular weight formulations a control of the release behaviour is not feasible by selection of the drug loading. However, this advantageously allows adjustment of the amount of triptorelin delivered per day. In the range between 2.5% and 10%, an Increase in loading is almost linearly linked to an increase in drug delivered per unit of time for formulations with hexPLA of 5,000 g/mol.

In conclusion, the release rate of low molecular weight polymer can be controlled by the drug loading, which directly influences the viscosity of the formulation. For higher molecular weights the effect of the loading on the release profiles decreases, allowing an adjustment of the daily delivered drug dose.

### Influence of the Molecular Weight on the Release

FIG. 8 compares the triptorelin release of three formulations containing the same amount of drug but having different molecular weights. The formulations with polymer of 1,500 g/mol showed biphasic release; the higher molecular weights of 2,500 g/mol and 5,000 g/mol show multiphase release behavior. The burst release was less pronounced for the 5,000 g/mol polymer in comparison to the 2,500 g/mol (and the 1,500 g/mol polymer) due to the increased rigidity of the polymer matrix, which retards the penetration of the buffer into the polymer. Accordingly, the molecular weight is directly linked to the degree of burst release, showing decreasing values for the burst release at higher molecular weights.

The 2,500 g/mol and 5,000 g/mol formulations appeared to show a “lag time” in the second phase after the Initial burst because the triptorelin close to the surface is dissolved relatively quickly. After time dependent degradation of the polymer on the surface the previously shielded core of the formulation comes into contact with the buffer solution and releases the active compound. Thus, in the second phase the “lag time” of the 5,000 g/mol polymer is prolonged in comparison to the 2,500 g/mol polymer, because more ester bonds need to be cleaved before the polymer degraded and the triptorelin can be released. Consequently, the release profile is shifted towards later time points for the higher molecular weight formulation. In the fourth phase the release appeared to decrease again for both molecular weights because the polymer spheres became smaller, reducing the surface and thus the interplay area between buffer and formulation.

In short, it is clear that the duration of release and the amount of burst are controllable by adjusting the molecular weight of the polymer suspensions.

### Conclusions

Formulations with polymer of 1,500 g/mol had a release of triptorelin largely influenced by the viscosity of the formulation, effectively by its loading. Higher molecular weight formulations release triptorelin independently of their drug loading; triptorelin release per day can hence be targeted by choice of loading. With increasing molecular weight and increasing drug loading the release mechanism apparently evolves from a viscosity driven mechanism to a polymer degradation driven release. Formulations with 5% and 10% Triptorelin in polymers of 2,500 g/mol and 5,000 g/mol had an advantageous release profile for a sustained release formulation of triptorelin for several months.

Thus the formulations of the invention may display continuous sustained-release over 3 months with a good correlation between polymer degradation and release. The release behavior is apparently controllable by adjusting the viscosity for low molecular weight formulations and by adjustment of the molecular weight for high molecular weight formulations. Formulations with 5% to 10% Triptorelin in polymer of 2,500 g/mol appear especially interesting because they have a long release period and an advantageous ratio between burst and sustained-release. For formulations of this polymer molecular weight the daily drug dose may be be adapted since the release is independent of the drug incorporation. Further, the suspensions were easily injectable and stable during storage at room temperature.

The results demonstrate the advantages of using a hexyl substituted polylactide polymer excipient for peptide (e.g. triptorelin) sustained-release delivery.

### EXAMPLE 6

### In Vivo Release of Triptorelin-hexPLA Formulations

Formulations with 5% and 10% Triptorelin in the polymer of 2,500 g/mol were evaluated in a 168 day pharmacokinetic study in male SPF Wistar Hannover rats, as shown in FIG. 9 (solid line=10% triptorelin, dashed line=5% triptorelin).

30 μL corresponding to 30 mg of the formulation with 10% Triptorelin were injected subcutaneously into 16 rats using Hamilton syringes. To apply equal amounts of triptorelin 60 μL were used of the formulations with 5% active substance in a second group of 16 animals. Both groups were divided into subgroups of 8 animals to allow alternating sampling. Dosing and sample analysis was similar to Example 2, as understood by those skilled in the art.

The animals were surveyed regarding reactions at the injection site, weight changes and other clinical signs. At each sampling point, 0.8 mL blood were taken into EDTA by retro-orbital bleeding under light CO₂/O₂ anesthesia. After 168 days the animals were sacrificed by carbon dioxide and the injection sites were optically investigated. The Triptorelin concentration in the plasma was determined by HPLC-MS, as described above.

The formulations were easily injectable, which is important for a convenient clinical use.

Local Tolerance

All animals remained healthy throughout the experiment, none showing weight-loss or other signs of pathological behaviour. Despite the liquid aggregate state of the formulation, it formed a depot at the Injection site due to the high lipophilicity and cohesion of the polymer. The formulation degraded at the site of injection and stayed localized for six months, allowing simple recovery of the formulation. The formulation showed excellent histocompatibility. No, or only minor, inflammation signs were observed, and any that did disappeared shortly after administration. At the endpoint of the in vivo study, the remaining formulation was not encapsulated by connective tissue containing macrophages. The absence of any encapsulation is advantageous for the polymer because degradation and release are more similar in vitro and in vivo.

The good general and local tolerability also demonstrates efficacy of the formulation as an injectable parenteral formulation.

The plasma levels of Triptorelin released from implant formulations with 5% and 10% drug loading in Wistar rats are summarized in FIG. 9 (solid line=10% triptorelin, dashed line=5% triptorelin). In order to administer equal amounts of drug, 60 mg of the formulation with 5% loading were injected and 30 mg of the 10% formulation. Both formulations displayed a similar plasma level profile, having a higher rate of release during the first seven days followed by constant plasma levels at around 3000 pg/mL. After 10 to 12 weeks, the plasma drug concentrations slowly decrease until the end of the study. However, during the entire 6 months (24 weeks), the drug levels permanently remained above the level of efficacy (100 pg/mL). The Initial higher drug levels are important for all GnRH-receptor agonists to stimulate all receptors and Induce rapid reduction of the testosterone levels. During the first four days, the active compound levels were higher in the animals with the formulation containing 5% triptorelin than with 10%, presumably due to the larger volume administered exhibiting more of the active compound on its surface. From week 2 to 10 both formulations released equal amounts of Triptorelin. Later on, the release rate decreased slightly faster for the 5% Triptorelin formulation. The plasma level profiles, resulting from a zero order drug release between week one and up to 12 weeks, suggest a controlled release mechanism modulated by the degradation of the polymer. Accordingly, these formulations have a matrix-controlled release in vivo. The studied formulations indicate plasma level profiles feasible for parenteral sustained-release formulations with application intervals of 6 months, and demonstrated potential for release periods of even longer, e.g. 9 to 12 months by appropriate adjustment of formulation parameters such as the molecular weight of the polymer and drug loading.

1. A pharmaceutical composition comprising a liquid, viscous, alkyl substituted polylactide; and a GnRH analog or a pharmaceutically acceptable salt or derivative thereof.

2. A pharmaceutical composition according to claim 1 wherein the liquid, viscous, alkyl substituted polylactide is a C₅-C₁₁ alkyl substituted polylactide.

3. A pharmaceutical composition according to claim 1 wherein the GnRH analog is a GnRH agonist.

4. A pharmaceutical composition according to claim 1 wherein the GnRH analog is triptorelin.

5. A pharmaceutical composition according to claim 1 wherein the liquid, viscous, alkyl substituted polylactide is a hexyl substituted polylactide.

6. A pharmaceutical composition according to claim 1 having a viscosity of 5 to 70 Pa×s, for example 5 to 65 Pa×s, for example 15 to 35 Pa×s, at a temperature of 20° C. and a shear rate of 100 1/s or less.

7. A pharmaceutical composition according to claim 1 comprising micronised triptorelin or pharmaceutically acceptable salt or derivative thereof.

8. A pharmaceutical composition according to claim 1 comprising particles which include triptorelin or a pharmaceutically acceptable salt or derivative thereof, the particles having an average particle diameter of 0.5 to 11 μm, for example 2.0 μm to 9.5 μm for example 3.5 μm to 5 μm.

9. A pharmaceutical composition according to claim 1 wherein the weight average molecular weight of the alkyl substituted polylactide is from 1,000 to 10,000 g/mol, preferably 1,200 to 7,500 g/mol.

10. A pharmaceutical composition according to claim 1 wherein the weight average molecular weight of the alkyl substituted polylactide (hexyl substituted polylactide) is from 1,000 to 7,500 g/mol.

11. A pharmaceutical composition according to claim 1 wherein the weight average molecular weight of the alkyl substituted polylactide (hexyl substituted polylactide) is 2,750 to 10,000 g/mol.

12. A pharmaceutical composition according to claim 1 wherein the weight average molecular weight of the alkyl substituted polylactide (hexyl substituted polylactide) is 2,000 to 6,000 g/mol, preferably 2,100 to 5,100 g/mol, preferably 2,200 to 3,000 g/mol.

13. A pharmaceutical composition according to claim 1 wherein the weight average molecular weight of the alkyl substituted polylactide (e.g. hexyl substituted polylactide) in the composition is 1,800 g/mol or greater.

14. A pharmaceutical composition according to claim 1 further comprising a plasticiser.

15. A pharmaceutical composition according to claim 1 further comprising 2 to 15% by weight triptorelin or a pharmaceutically acceptable salt or derivative thereof, preferably 4 to 12% by weight triptorelin or a pharmaceutically acceptable salt or derivative thereof, more preferably 5 to 10% by weight triptorelin or a pharmaceutically acceptable salt or derivative thereof.

16. A pharmaceutical composition according to claim 1 formed by a method comprising micronising the triptorelin or pharmaceutically acceptable salt or derivative thereof and mixing with the alkyl substituted polylactide.

17. A pharmaceutical composition according to claim 1 formed by a single step of cryomilling the triptorelin or pharmaceutically acceptable salt or derivative thereof with the alkyl substituted polylactide.

18. A process for preparation of a pharmaceutical composition comprising a liquid, viscous, alkyl substituted polylactide (e.g. a C₅-C₁₁ alkyl substituted polylactide); and triptorelin or a pharmaceutically acceptable salt or derivative thereof; the process comprising micronising the triptorelin or pharmaceutically acceptable salt or derivative thereof and mixing the triptorelin or pharmaceutically acceptable salt or derivative thereof with the polylactide.

19. A process according to claim 18 comprising a single step of micronising the triptorelin or pharmaceutically acceptable salt or derivative thereof together with the polylactide.

20. A pharmaceutical composition, for example a room temperature stable pharmaceutical composition, comprising a liquid, viscous, alkyl substituted polylactide (e.g. a C₅-C₁₁ alkyl substituted polylactide, e.g. a hexyl substituted polylactide); and a GnRH analog or a pharmaceutically acceptable salt or derivative thereof.

21. A pharmaceutical composition according to claim 20 wherein the weight average molecular weight of the alkyl substituted polylactide (e.g. hexyl substituted polylactide) in the composition is 1,800 g/mol or greater.

22. A pharmaceutical composition according to claim 1 for use in the treatment of hormone-responsive cancers such as breast cancer or prostate cancer; in the management of endometriosis, female infertility and uterine fibroids; or in treatment of precocious puberty.

23. Use of a pharmaceutically effective amount of a composition comprising a liquid, viscous, alkyl substituted polylactide (e.g. a C₅-C₁₁ alkyl substituted polylactide, e.g. a hexyl substituted polylactide); and a GnRH analog or a pharmaceutically acceptable salt or derivative thereof in the manufacture of a medicament for the treatment of hormone-responsive cancers such as breast cancer or prostate cancer; endometriosis, female infertility, uterine fibroids; and/or precocious puberty.

24. A method of treatment of hormone-responsive cancers such as breast cancer or prostate cancer; a method of treatment or management of endometriosis, a method of treatment of female infertility, a method of treatment of uterine fibroids; and/or a method of treatment of precocious puberty; comprising a step of administering to a patient in need thereof a pharmaceutically effective amount of a composition comprising a liquid, viscous, alkyl substituted polylactide (e.g. a C₅-C₁₁ alkyl substituted polylactide, e.g. a hexyl substituted polylactide); and a GnRH analog or a pharmaceutically acceptable salt or derivative thereof.

25. A method according to claim 24 comprising a further step of warming the composition to above 25° C. prior to administration to the patient.
